(19) United States
(12) Patent Application Publication
(10) Pub. No.: US 2004/0242970 A1
(43) Pub. Date: Dec. 2, 2004

(54) Methods and apparatus for retracting the soft tissues of the mouth

(76) Inventor: Lance S. Burns, Glendale, AZ (US)

Correspondence Address: Daniel R. Pote, SNELL & WILMER LLP, One Arizona Center, 400 East Van Buren, Phoenix, AZ, 85374-2202, US

(21) Appl. No.: 10/446,765

(22) Filed: May 28, 2003

(51) Int. Cl. A61B 1/32

(52) U.S. Cl. 600/238

(57) ABSTRACT

A retractor is configured to increase the oral aperture of an individual and thereby assist in visualization of the vocal cords (and other features of the individual's mouth and throat) to facilitate intubation. The retractor generally comprises a mouthpiece configured to grasp a soft tissue of the individual's mouth (e.g., at the corner of the mouth), an anchor component configured to removeably attach to the head of the individual (e.g., via the individual's ear canal), and a collapsable member coupled between the mouthpiece and the anchor component, wherein the collapsable member is configured to pull the mouthpiece toward the anchor such that the lips of the individual are retracted.

### BACKGROUND OF THE INVENTION

1. Technical Field

This invention generally relates to retractors for use in the medical field and, more particularly, to a retractor configured to retract the soft tissues of the mouth.

2. Background Information

During the course of providing medical attention to an individual, it is often necessary to position a tube within a hollow region or cavity of that individual's body. Such a procedure is generally referred to as intubation.

One intubation procedure that is particularly important, and one that is often performed in an emergency room context, involves the placement of a tube within the mouth and trachea to provide air to the lungs. This intubation is difficult in that it is non-trivial to visualize the position of the vocal cords when placing the tube within the body. More particularly, the soft tissues of the mouth and the endotracheal tube itself generally obscure the vocal cords, making it difficult to properly place the tube.

While it is possible to instruct an assistant to manually retract the lips and/or cheeks of the individual being intubated, such a procedure involves the use of valuable personnel for the task and, depending upon the proficiency of the assistant, may give inconsistent results.

Prior art lip retraction devices are generally unsatisfactory in that they are designed to achieve much different goals, e.g., exposure of the teeth and/or gums for general dentistry and oral surgery.

Accordingly, there is a need for a system which overcomes these and other limitations of the prior art.

### BRIEF SUMMARY OF THE INVENTION

The present invention provides a retractor configured to increase the oral aperture of an individual and thereby assist in visualization of the vocal cords (and other internal features of the individual's mouth and throat) to facilitate intubation. The retractor generally comprises a mouthpiece configured to grasp a soft tissue of the individual's mouth (e.g., at the corner of the mouth), an anchor component configured to removeably attach to the head of the individual (e.g., via the individual's ear canal), and a collapsable member coupled between the mouthpiece and the anchor component, wherein the collapsable member is configured to pull the mouthpiece toward the anchor such that the lips of the individual are retracted.

In accordance with one embodiment of the present invention, the collapsable member includes an inner component configured to slideably engage an outer component. A ratchet feature is preferably included to allow the collapse of the inner component with respect to the outer component and to substantially prevent expansion of the inner component with respect to the outer component.

In accordance with a further aspect of the present invention, one or more thumb rests, loops, of other such features are mechanically coupled to the mouthpiece (or the mouthpiece end of the collapsable member) and/or the anchor component.

In accordance with yet another aspect of the present invention, a light source is attached to the mouthpiece to assist in illuminating the interior of the individual's mouth.

### BRIEF DESCRIPTION OF THE DRAWINGS

The subject invention will hereinafter be described in conjunction with the appended drawing figures, wherein like numerals denote like elements, and:

FIG. 1 presents a retractor in accordance with one embodiment of the present invention;

FIG. 2 depicts the retraction of the soft tissues near the corner of the mouth;

FIG. 3 shows a rear view of a mouthpiece in accordance with one embodiment of the present invention;

FIG. 4 shows a ear-piece anchor component in accordance with one embodiment of the present invention;

FIG. 5 depicts a collapsable member in accordance with an alternate embodiment of the present invention;

FIGS. 6 and 7 depict an anchor component supported by the back of the ear; and

FIG. 8 shows a retractor in accordance with an alternate embodiment of the present invention wherein the mouthpiece includes a light source.

### DETAILED DESCRIPTION

The present invention provides methods and apparatus for a retractor configured to increase the oral aperture of an individual to facilitate intubation. In accordance with various aspects of the present invention, a retractor comprises a mouthpiece configured to grasp a soft tissue of the individual's mouth, an anchor component configured to removeably attach to the head of the individual, and a collapsable member coupled between the mouthpiece and the anchor component, wherein the collapsable member is configured to pull the mouthpiece toward the anchor such that the lips of the individual are retracted.

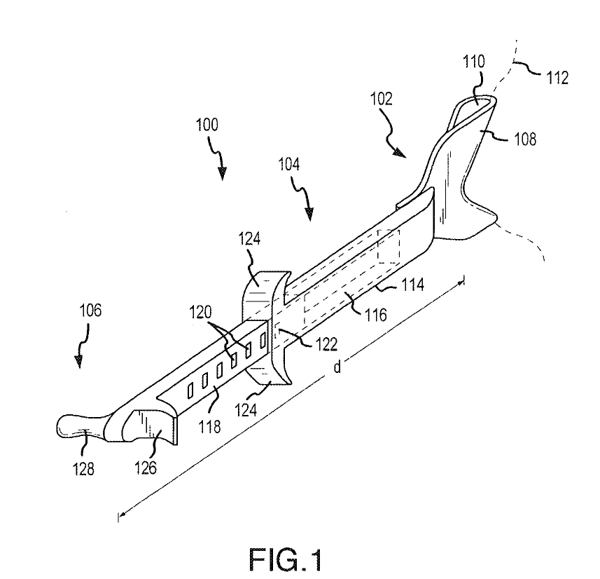

With reference to FIG. 1, a retractor 100 in accordance with one embodiment of the present invention comprises an anchor component 106, a collapsable member 104, and a mouthpiece 102. Mouthpiece 102 preferably includes a grasping component 108 having an inner diameter 110 configured to “hook”, grasp, or otherwise accommodate the lips 112 at the corner of the individual's mouth. Grasping component 108 is rigidly or removeably attached to collapsable member 104, which itself is rigidly or removeably attached to anchor component 106, while anchor component 106 is configured to attach to the individual's head using any convenient method. In the illustrated embodiment, for example, anchor component 106 includes a protruding component 128 configured to fit within the ear canal of the individual. In this way, when collapsable member 104 is manipulated to bring mouthpiece 102 closer to anchor component 106, anchor component 106 remains firmly imbedded within the ear canal of the individual while the soft tissue 112 at the corner of the individual's mouth is retracted by mouthpiece 102, thus increasing the oral aperture and facilitating intubation.

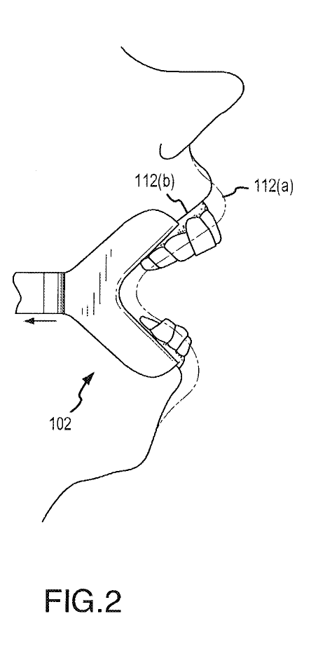

Referring briefly to FIG. 2, when the retractor is first moved into place such that mouthpiece 102 engages the lips at the corner of the mouth, the lips have the general configuration indicated by reference numeral 112(a). When the retractor is activated, however, mouthpiece 102 moves back (i.e., toward the back of the individual's head) such that the original position soft tissues are retracted to a subsequent position generally indicated by reference numeral 112(b).

In accordance with one embodiment of the present invention, shown in FIG. 1, collapsable member 104 includes an inner component 118 configured to slideably engage an outer component 114, for example, via an axial cavity 116 which receives inner component 118. A ratchet feature (120 and 122) is configured to allow the collapse of inner component 118 with respect to outer component 114 and to substantially prevent expansion of inner component 118 with respect to outer component 114. In the illustrated embodiment, multiple protrusions 120 are provided at suitable intervals along an outer surface of inner component 118 and a matching cavity 122 is provided within an inner surface of outer component 114. Consistent with the desired ratchet effect, protrusions 120 preferably have a conventional right triangular shape such that they move inward and engage cavity 122 easily but are difficult to remove, thus substantially preventing expansion of member 104. Those skilled in the art will recognize that the dimensions and position of protrusions 120 may be selected to withstand any desired tensile stress likely to be induced during retraction of lips 112. Thus, the care-giver may release his or her grasp on the retractor and it will remain in position by virtue of the tension formed between the lips 112 and anchor component 106 which, in the illustrated embodiment, is firmly imbedded in the ear canal.

While the inner component 118 and 114 are shown as generally rectilinear, it will be appreciated that these components may be rounded or have any suitable curvilinear shape. In addition, it is not necessary that these components move coaxially with respect to one another; they may be located side-by-side, on tracks, or in any other suitable arrangement.

Collapsable member 104 may be configured to provide any desired range of lengths d between mouthpiece 102 and anchor component 106, both in the collapsed state and uncollapsed state. For example, in one embodiment, the retractor has a maximum un-collapsed distance d between about 4.0 inches and 6.0 inches, preferably about 4.5 inches, and a minimum collapsed distance d between about 1.5 and 4.0 inches, preferably about 2.5 inches. That is, collapsable member 104 has a suitable travel of between about 1.0 and 3.0 inches, preferably about 2.0 inches.

In accordance with a further aspect of the present invention, one or more thumb or finger rests, loops, of other such features are mechanically coupled to the mouthpiece (or the mouthpiece end of the collapsable member) and/or the anchor component to facilitate manipulation by the doctor or nurse. For example, as shown in FIG. 1, a set of finger rests 124 are attached to outer component 114 (and thus mechanically coupled to mouthpiece 102) and a thumb rest 126 is connected to anchor component 106. In the illustrated embodiment the thumb and finger rests are shown as wide protrusions having a generally arcuate surface intended to accommodate the thumb and or fingers of the care-giver. Those skilled in the art, however, will recognize that a variety of different thumb and finger rests may be used in connection with the present invention, including rests shaped as rings, pegs, or any other suitable shape. Moreover, it may be appropriate to place finger rests on the end corresponding to anchor component 106 and a thumb rest on the end corresponding to the mouthpiece 102.

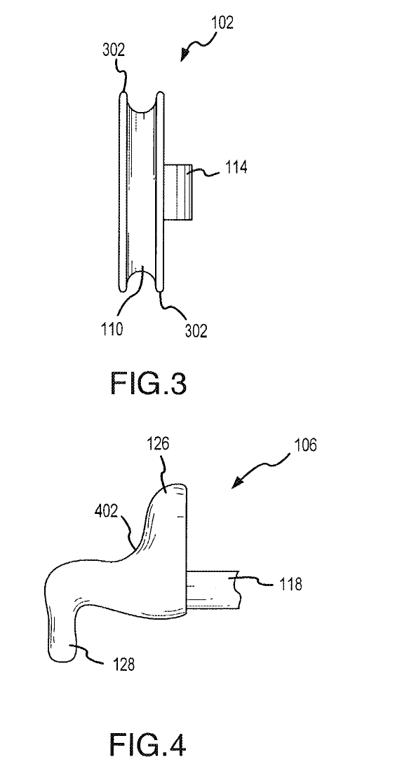

FIG. 3 shows a rear view of a mouthpiece 102 in accordance with one embodiment of the present invention. As shown, mouthpiece 102 preferably includes a smooth inner diameter 110 configured to accommodate the lips of an individual. The edges 302 of mouthpiece 102 are preferably rounded to facilitate insertion of the device and to reduce the occurrence of cuts or abrasions resulting from insertion and use of the retractor.

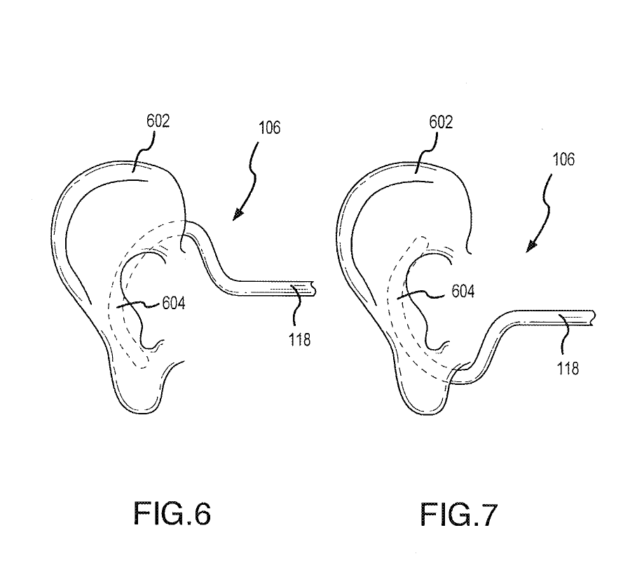

FIG. 5 shows a collapsable member 104 in accordance with another embodiment of the present invention wherein the inner component 118 is coupled to outer component 114 via a suitable spring 502. In this embodiment, collapsable member 104 is normally in a collapsed position, but can be expanded by the care-giver to engage the individual's lips and to anchor the anchor component before letting go of the retractor.

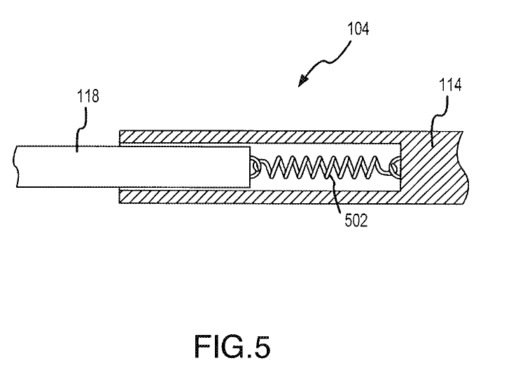

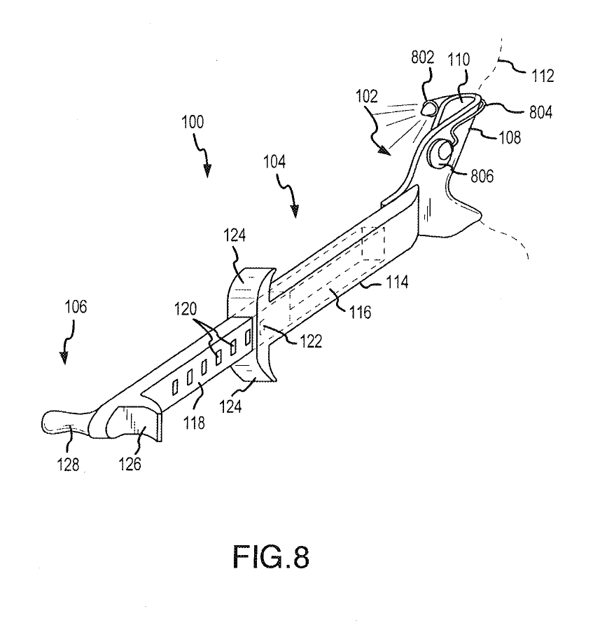

FIG. 4 shows an anchor component 106 in accordance with one embodiment of the present invention wherein a thumb rest 126 has a generally smooth surface 402 opposite protruding component 128, which fits within the ear canal of the individual. It will be appreciated that the present invention contemplates any number of different anchor components. As shown in FIGS. 6 and 7, for example, anchor component 106 might include a generally loop-shaped structure configured to hook to the back of the ear's exterior either from above the ear (FIG. 6) or below the ear (FIG. 7). Anchor component 106 may have any number of other configurations, and may be anchored, for example, behind the individual's head, on the sides of the individual's head, or on any other suitable location on the individual's body. In one embodiment, for example, a pair of mouthpieces 102 are connected by an extended elastic collapsable member 104 that fits behind the individual's head.

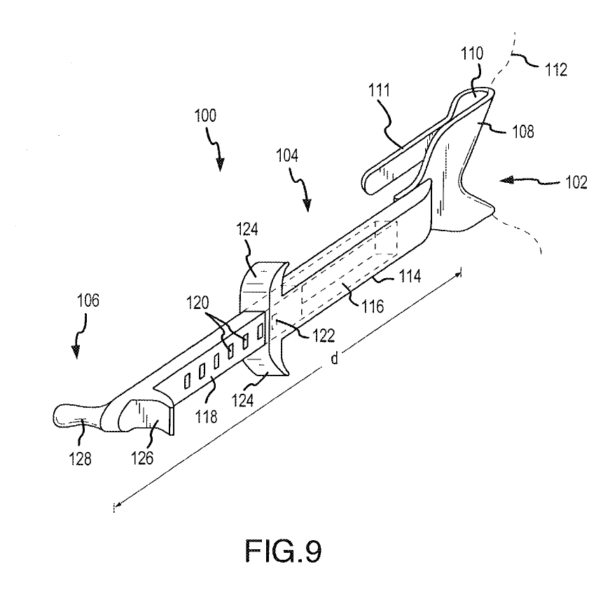

In accordance with yet another aspect of the present invention, a light source is attached to the mouthpiece to assist in illuminating the interior of the individual's mouth. Referring to FIG. 8, for example, a light source 802 is configured to shine within the buccal (interior) of the mouth when the device is deployed. Light source 802 (e.g., an LED, incandescent bulb, fiber-optic component, or the like) is electrically connected (via conductors 804) to a suitable power source, e.g., a small battery 806 provided on component 806 or any other suitable surface of the device.

A switch (not shown) may be provided to allow the caregiver to selectively turn on light source 802 when appropriate. Alternatively, in the case where the retractor is generally disposable, it may be advantageous to provide a disposable film or paper segment between one electrode of battery 806 and the corresponding clip or wire which is to make contact with that electrode. In this way, the disposable film may be pulled away before use, allowing the light to illuminate and ultimately burn-out after disposal. In an alternate embodiment, a suitable switch is configured such that light source 802 illuminates only when collapsable member 104 is in a collapsed position.

In general, the retractor of the present invention may be designed as a “single-use”, disposable unit, or as an entirely reusable unit. In this regard, while the retractor has been generally illustrated as comprising two or more separate components, the present invention contemplates the use of a single, simply integrated component that serves the same purpose. For example, collapsable component 104 may consist of a simple plastic loop integrated with an anchor component on one end and a mouthpiece at the other end with integrated finger and thumb loops to facilitate elastic expansion of the retractor during deployment.

Alternatively, one or more components may be disposable while others are reusable. For example, it may be advantageous to reuse collapsable member 104 while providing replaceable (i.e., disposable) anchor components and mouthpieces. In such a case, mouthpiece 102 and/or anchor component 106 are preferably configured to removeably attach to the respective ends of collapsable member 104.

Collapsable member 104, anchor component 106, and mouthpiece 102 are fabricated from any suitable material or materials, including various plastics, ceramics, metals, and the like. Mouthpiece 102 and anchor component 106, for example, preferably comprise a relatively smooth plastic. In the event any of these components are intended to be reused and sterilized, however, it is preferably that they comprise titanium, stainless steel, or any other such material suitable for the sterilization process.

In summary, what has been disclosed is a retractor configured to increase the oral aperture of an individual and thereby assist in visualization of the vocal codes (and other features of the individual's mouth and throat) to facilitate intubation. The illustrated embodiments, however, are not intended to limit the various structures and applications of the retractor. Various modifications in the selection, design, and arrangement of the various components and steps discussed herein may be made without departing from the scope of the invention as set forth in the appended claims.

What is claimed is:

1. A retractor for increasing the oral aperture of an individual to facilitate intubation, said retractor comprising:
a mouthpiece configured to grasp a soft tissue of the individual's mouth;
an anchor component configured to removeably attach to the head of the individual;
a collapsable member coupled between said mouthpiece and said anchor component, said collapsable member configured to pull said mouthpiece toward said anchor.

2. The retractor of claim 1, wherein said mouthpiece is configured to grasp the soft tissue at a corner of the individual's mouth.

3. The retractor of claim 1, wherein said mouthpiece has an inner radius to accommodate the lips of the individual.

4. The retractor of claim 1, wherein said collapsable member includes:
an inner component configured to slideably engage an outer component;
a ratchet feature configured to allow the collapse of said inner component with respect to said outer component and to substantially prevent expansion of said inner component with respect to said outer component.

5. The retractor of claim 1, further including a thumb rest mechanically coupled to said anchor component and at least one finger rest mechanically coupled to said mouthpiece.

6. The retractor of claim 1, further including a thumb rest mechanically coupled to said mouthpiece and at least one finger rest mechanically coupled to said anchor component.

7. The retractor of claim 1, wherein said collapsable member includes a spring configured to provide tension between said anchor component and said mouthpiece.

8. The retractor of claim 1, wherein said anchor component is configured to be removeably inserted with the individual's ear canal.

9. The retractor of claim 1, wherein said anchor component is configured to grasp the back of the individual's ear.

10. The retractor of claim 1, further including a light source attached to said mouthpiece and configured to illuminate the interior of the individual's mouth.
